(19) United States
(12) Patent Application Publication
(10) Pub. No.: US 2013/0303745 A1
(43) Pub. Date: Nov. 14, 2013

(54) Synthesis of oligonucleotides

(71) Applicants: Meinolf LANGE, Bielefeld (DE); Andreas HOHLFELD, Porta Westfalica (DE); Andreas SCHONBERGER, Muden/Aller (DE); Christina KIRCHHOFF, Halle/Westfalen (DE); Olaf GROSSEL, Halle/Westfalen (DE)

(72) Inventors: Meinolf LANGE, Bielefeld (DE); Andreas HOHLFELD, Porta Westfalica (DE); Andreas SCHONBERGER, Muden/Aller (DE); Christina KIRCHHOFF, Halle/Westfalen (DE); Olaf GROSSEL, Halle/Westfalen (DE)

(21) Appl. No.: 13/941,272

(22) Filed: Jul. 12, 2013

Related U.S. Application Data
(63) Continuation of application No. 12/839,078, filed on Jul. 19, 2010, now abandoned.
Application No. 12/839,078 is a continuation of application No. 12/599,931, now abandoned.
(60) Provisional application No. 60/939,480, filed on May 22, 2007.

(51) Int. Cl. C07H 21/00

(52) U.S. Cl. CPC C07H 21/00
USPC 536/25.34

(57) ABSTRACT

A method for preparing an oligonucleotide comprising the steps of synthesizing a phosphoramidite by reacting a hydroxyl-containing compound of formula (A) with a phosphitylating agent in the presence of an activator compound of formula (I), to prepare a phosphitylated compound, then coupling the phosphitylated compound without isolation with a second compound having the formula (A), wherein R₅, R₃, R₂, B are independently selected, but have the same definition as above in the presence of an activator II selected from the group of imidazole, imidazolium salts, and mixtures thereof, which are improved activators over activators disclosed in related art.

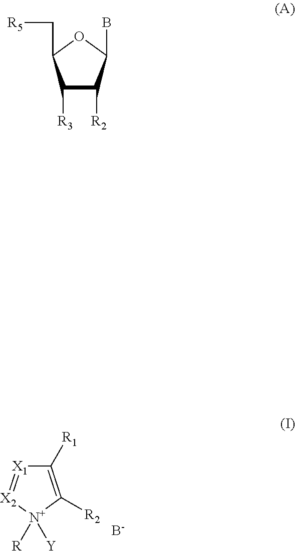

### CROSS-REFERENCE TO RELATED APPLICATIONS

This application is a continuation of U.S. application Ser. No. 12/839,078 filed Jul. 19, 2010, incorporated herein by reference in its entirety for all purposes, which is a continuation of a U.S. application Ser. No. 12/599,931, abandoned, incorporated herein by reference in its entirety for all purposes, which is a U.S. National Stage entry under 35 U.S.C. §371 of International Application PCT/EP2007/062660 filed Nov. 21, 2007, which claims priority to U.S. Provisional Application 60/939,480 filed May 22, 2007, which is itself incorporated herein by reference in its entirety for all purposes.

### FIELD OF THE INVENTION

The present invention relates to methods for preparing oligonucleotides.

### BACKGROUND OF THE INVENTION

Oligonucleotides are key compounds in life science having important roles in various fields. They are for example used as probes in the field of gene expression analysis, as primers in PCR or for DNA sequencing.

Furthermore, there are also a number of potential therapeutic applications including i.e. antisense oligonucleotides.

The growing number of applications requires larger quantities of oligonucleotides, therefore, there is an ongoing need for developing improved synthetic method.

For a general overview, see for example “Antisense—From Technology to Therapy” Blackwell Science (Oxford, 1997).

One prominent type of building blocks in the synthesis of oligonucleotides are phosphoramidites; see for example S. L. Beaucage, M. H. Caruthers, Tetrahedron Letters 1859 (1981) 22. These phosphoramidites of nucleosides, deoxyribonucleosides and derivatives of these are commercially available. In normal solid phase synthesis 3′-O-phosphoramidites are used but in other synthetic procedures 5′-O and 2′-O-phosphoramidites are used, too. One step in the preparation of these nucleosides phosphoramidites is the phosphitylating of the (protected) nucleosides. After phosphitylation the prepared amidites are normally isolated by using cost intensive separation methods e.g. chromatography. After isolation the sensitive amidites have to be stocked under special conditions (e.g. low temperature, water-free). During storage the quality of the amidites may be reduced by a certain degree of decomposition and hydrolysis. Both side reactions can appear and the results are detectable. Most commonly, the hydroxyl group and amino groups and other functional groups present in the nucleoside are protected prior to phosphitylating the remaining 3′-, 5′- or 2′-O hydroxyl group.

These phosphoramidites are then coupled to hydroxyl groups of nucleotides or oligonucleotides. The usage of the isolated amidite can also result in a partial hydrolysis during the amidite coupling.

Phosphoramidites are expensive compounds. Typical prices for deoxyamidites are in the range of [symbol] 40.00 per g. The corresponding RNA building blocks are even more expensive.

WO 2006/094963 discloses a method for preparing oligonucleotides comprising the steps of synthesizing a phosphoramidate in the presence of an activator I and coupling in the presence of an activator II. As activators II tetrazole derivatives, pyridinium salts and 4,5-dicyanoimidazole are described. Summary of the invention

### SUMMARY OF THE INVENTION

It is an object of the present invention to provide a method for preparing oligonucleotides overcoming at least some of the drawbacks of prior art.

The present patent application is related to an improvement of the invention disclosed in the patent application WO 2006/094963 the content of which is incorporated by reference into the present patent application.

The invention concerns in particular a method for preparing an oligonucleotide according to claim 1 of WO 2006/094963 with an improved activator II.

In one embodiment, the invention provides a method for preparing an oligonucleotide comprising the steps of
a) providing a hydroxyl containing compound having the formula:

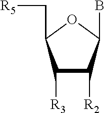

wherein
B is a heterocyclic base
and
i) R₂ is H, a protected 2′-hydroxyl group, F, a protected amino group, an O-alkyl group, an O-substituted alkyl, a substituted alkylamino or a C4′-O2′ methylene linkage;
   R₃ is OR′₃, NHR″₃, NR″₃R′″₃, wherein R′₃ is a hydroxyl protecting group, a protected nucleotide or a protected oligonucleotide, wherein R″₃ and R′″₃ are independently amine protecting groups; and R₅ is OH;
or
ii) R₂ is H, a protected 2′-hydroxyl group, F, a protected amino group, an O-alkyl group, an O-substituted alkyl, a substituted alkylamino or a C4′-O2′ methylene linkage;
   R₃ is OH; and
   R₅ is OR′₅ and R′₅ is a hydroxyl protecting group, a protected nucleotide or a protected oligonucleotide;
or
iii) R₂ is OH;
   R₃ is OR′₃, NHR″₃, NR″₃R′″₃, wherein R′3 is a hydroxyl protecting group, a protected nucleotide or a protected oligonucleotide, wherein R″₃ and R′″₃ are independently amine protecting groups; and
   R₅ is OR′₅ and R′₅ is a hydroxyl protecting group, a protected nucleotide, or a protected oligonucleotide; and
b) reacting said compound with a phosphitylating agent in the presence of an activator having the formula I (activator I)

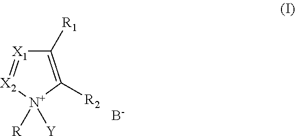

wherein
R=alkyl, cycloalkyl, aryl, aralkyl, heteroalkyl, or heteroaryl;
R₁, R₂=either H or form a 5-membered to 6-membered ring together X₁, X₂=independently either N or CH;
Y=H or Si(R₄)₃, with R₄=alkyl, cycloalkyl, aryl, aralkyl, heteroalkyl, or heteroaryl;
B=deprotonated acid;
to prepare a phosphitylated compound;
c) reacting said phosphitylated compound without isolation with a second compound having the formula

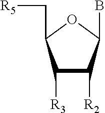

wherein R₅, R₃, R₂, B are independently selected, but have the same definition as above
in the presence of an activator II selected from the group consisting of imidazole and imidazolium salts.

### BRIEF DESCRIPTION OF THE DRAWINGS

FIG. 1 shows a reaction scheme according to the invention.

FIG. 2 shows another reaction scheme with imidazole according to the invention, wherein R1 (CH₂—OH) and R2 (CH₂—OH) represent (oligo-)nucleosides or -nucleotides.

### DETAILED DESCRIPTION OF THE INVENTION

“Imidazole” is an unsubstituted heterocyclic compound; the IUPAC name is 1,3-diazole or 1,3-diazocyclopenta-2,4-diene.

“Imidazolium” is a protonated form of the imidazole defined above. The aforesaid activators II are highly efficient for initiating the reaction of step (c) and are advantageous compared to activators II specifically disclosed in WO 2006/094963, in particular as far as industrial safety and protection of the environment is concerned.

According to the invention the phosphitylated compound is prepared by phosphitylating the hydroxyl group of a nucleoside, a nucleotide or an oligonucleotide by using activators having formula I which are preferably derivates of imidazole.

Without purification or isolation, the prepared sensitive phosphoramidite is coupled to hydroxyl groups of nucleosides, nucleotides or oligonucleotides in the presence of an activator II, different from activator I. There is no isolation of the prepared phosphoramidite, no separation of the amidite from activator I. Preferably the reaction is continued in the same reaction vessel. Activator II can be used in the presence of activator I.

The prior art activators for amidite coupling have a high reactivity for the activation of the amidite function. Using such an activator for phosphitylation produces also a certain degree of “overreaction” (e.g. 3′-3′ by-product). To overcome this and other problems the reactivity of the activator is modulated. In this case the reaction will stop selectively on the amidite level substantially free of by-products, such as 3′-3′-byproduct. Only this result (in-situ generation of the amidite) allows to continue the entire approach by starting with the amidite coupling.

The activator II has the ability to induce the coupling step. After addition of the activator II, the amidite will start with the amidite coupling. As activator compounds, imidazole and imidazolium salts are suitable, i.e. salts of imidazole with an acid, preferably a strong acid. Suitable acids are, for example, trifluoroacetate, triflate, dichloracetate, mesyl, tosyl, o-chlorophenolate.

Acids with a pKa below 4.5 are preferred for making imidazole salts.

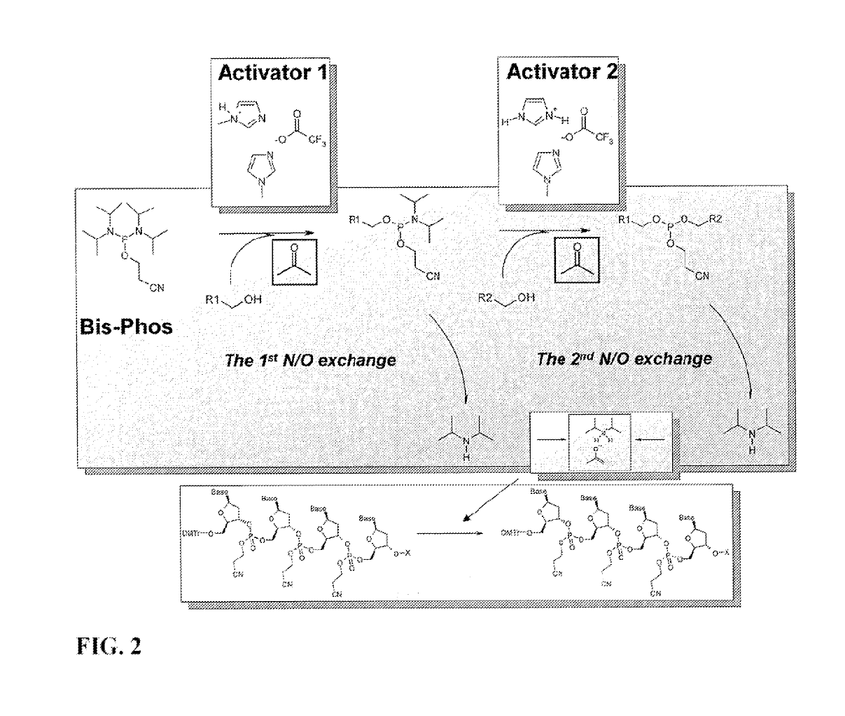

In one embodiment said activator is a protonated N-1-(H)imidazole. Counterions are generally as described in the WO 2006/094963. Trifluoroacetate is preferred as counterion. A particularly preferred reaction scheme with imidazole is shown in FIG. 2, wherein R1 (CH₂—OH) and R2 (CH₂—OH) represent (oligo-)nucleosides or -nucleotides.

The imidazole or imidazolium may be used in combination with other activators II, e.g. those disclosed in WO 2006/094963.

In a second aspect, said activator is tetrazole-poor. “Tetrazole” is understood to denote in particular the tetrazole compounds described in WO 2006/094963. Tetrazole-poor is understood to denote a quantity of tetrazole in the solution which is less than 1 mole per mole of hydroxyl containing compounds, as described in claim 1 of WO 2006/094963. This quantity is preferably less than 0.5 mole per mole of hydroxyl containing compounds and more preferably less than 0.1 mole per mole of hydroxyl containing compounds. In this aspect, said activator is preferably substantially free or totally free of tetrazole. Preferred activators in the second aspect are the activators according to the first aspect.

Preferred solvents in both aspects are C—H acidic solvents, in particular those containing a carbonyl group. Such solvents can be selected for example, from esters such as ethyl acetate or ethyl acetoacetate and ketones. Acetone is preferred.

The present invention covers inter alia a process according to claim 1 of WO 2006/094963, wherein activator II is an imidazole having an N⁰—H bond.

Preferably, the imidazole is protonated N-1-(H)imidazole.

The present invention covers further a process according to claim 1 of WO 2006/094963, wherein activator II is tetrazole-poor.

Preferably, the activator II is an imidazole having a N⁰—H bond, preferably protonated N-1-(H)imidazole.

After coupling, typically oxidation (PO formation) or sulfurisation (PS formation) are used. For the PO formation the peroxide approach is preferred. It is possible to perform this reaction without any extraction steps (iodine oxidation requires a few extraction steps).

In the case of sulfurisation, it is possible to use every known reagent for sulfurisation (i.e. PADS, S-Tetra, beaucage). A preferred reagent for PS formation is sulphur. The difference of production cost is in favour of the use of sulphur.

In one embodiment, the reaction may be in the presence of acetone.

The phosphitylating agent can either be used in a more or less equimolar ratio compared to the hydroxyl groups of the hydroxyl containing compound.

In a further embodiment, it can be used in an excess, e.g. 3 to 5 mol/mol of hydroxyl groups in the hydroxyl containing compound.

In one further preferred embodiment, a polymeric alcohol is added after step b) of claim 1. Suitable polymeric alcohols include polyvinylalcohol (PVA), commercially available as PVA 145000 from Merck, Darmstadt. Preferred are macroporous PVA with a particle size >120 μm (80%). Also membranes with hydroxyl groups or other compounds able to form enols are suitable.

The activator I can be used stoichiometrically, catalytically (from 3 to 50 mole %, preferably from 10 to 30 mole %) or in excess.

In a preferred embodiment, the activator I has a formula selected from the group consisting of

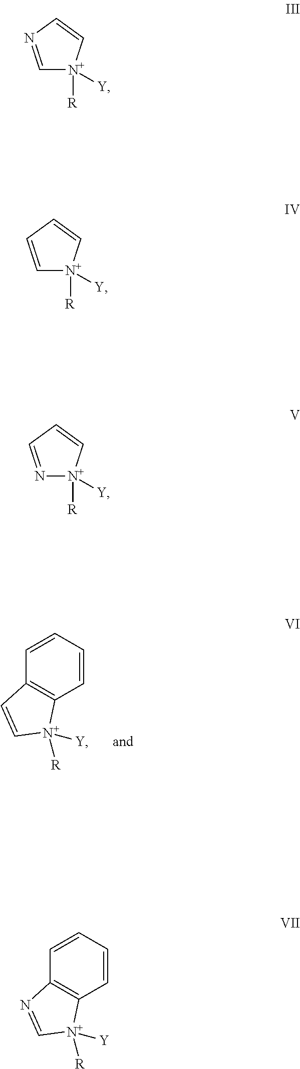

wherein
Y is H or Si(R₄)₃, with R₄=alkyl, cycloalkyl, aryl, aralkyl, heteroalkyl, or heteroaryl;
B=deprotonated acid; and
R is methyl, phenyl or benzyl.

The preparation of these activators is for example described in Hayakawa et al, J. Am. Chem. Soc. 123 (2001) 8165-8176.

In one embodiment the activator is used in combination with an additive. Additives can be selected from the unprotonated form of the compounds having formula I and other heterocyclic bases, for example pyridine. Suitable ratios between the activator and the additive are 1:1 to 1:10.

In one preferred embodiment, the activator can be prepared following an “in situ” procedure. In this case the activator will not be isolated, which resulted in improved results of the reaction. Hydrolysis or decomposition of the target molecule is suppressed.

For a high yielding phosphitylation in 3′- and/or 5′-position of oligonucleotides (di, tri, tetra, penta, hexa, hepta and octamers), the in-situ preparation of the activator and the combination with an additive is preferred.

As described above phosphitylating is especially useful in the synthesis of oligonucleotides and the building block phosphoramidites. Therefore, in a preferred embodiment, the hydroxyl containing compound comprises a sugar moiety for example a nucleoside or an oligomer derived therefrom. Such nucleosides are for example adenosine, cytidine, guanosine and uridine, 2′-deoxyadenosine, 2′-deoxyguanosine, 2′-deoxythymidine, 2′-deoxycytidine and derivatives thereof, optionally comprising protective groups.

Normally, they will be suitably protected on their heterocyclic functionality and on their hydroxyl bearing groups except of the one that should be phosphitylated. Typically, dimethoxytrityl, monomethoxytrityl or tbutyldimethyl-silyl (TBDMS) are used as protective groups for the 5′OH-group, allowing phosphitylation of the 3′-OH group. Further possible groups are phosphatesters and H-phosphonates, see for example

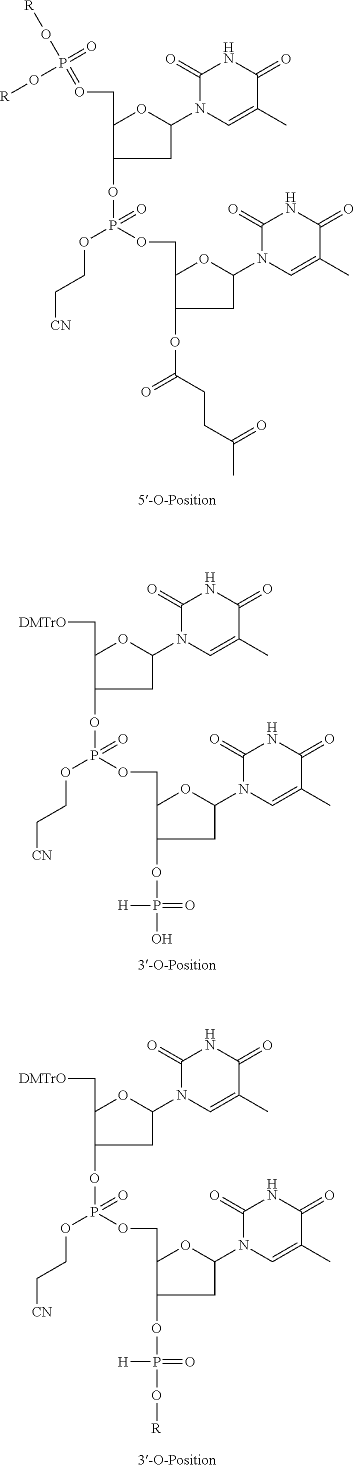

For phosphate ester and phosphodiester, R can be selected from alkyl, aryl, alkylaryl. Phenyl is preferred.

Further hydroxyl protecting groups for 5′, 3′ and 2′ are well-known in the art, e.g. TBDMS.

In general, the phosphitylating agent can be the same as in phosphitylating reactions using 1-H-tetrazole.

In a preferred embodiment, it has the formula

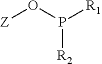

wherein Z represents a leaving group e.g., —CH₂CH₂CN, —CH₂CH═CHCH₂CN, para-CH₂C₆H₄CH₂CN, —(CH₂)₂₋₅N(H)COCF₃, —CH₂CH₂Si(C₆H₅)₂CH₃, or
—CH₂CH₂N(CH₃)COCF₃ and R₁ and R₂ are independently secondary amino groups N(R₃)₂, wherein R₃ is alkyl having from 1 to about 6 carbons; or R₃ is a heterocycloalkyl or heterocycloalkenyl ring containing from 4 to 7 atoms, and having up to 3 heteroatoms selected from nitrogen, sulfur, and oxygen.

A typical phosphytilating agent is 2-cyanoethyl-N,N,N′,N′-tetraisopropylphosphorodiamidite.

Other preferred phosphitylating reagents are oxazaphospholidine derivatives as described in N. Ok et al., J. Am. Chem. Soc. 2003, 125, 8307 to 8317 incorporated by reference. This phosphitylating agent allows the synthesis of oligonucleotides wherein the internucleotide bond can be converted to phosphorthioates in a stereo selective manner. Such diastereoselective synthesized internucleotidic phosphothioate linkages have promising impact on the use of phosphorthioates as antisense drugs or immunstimulating drugs.

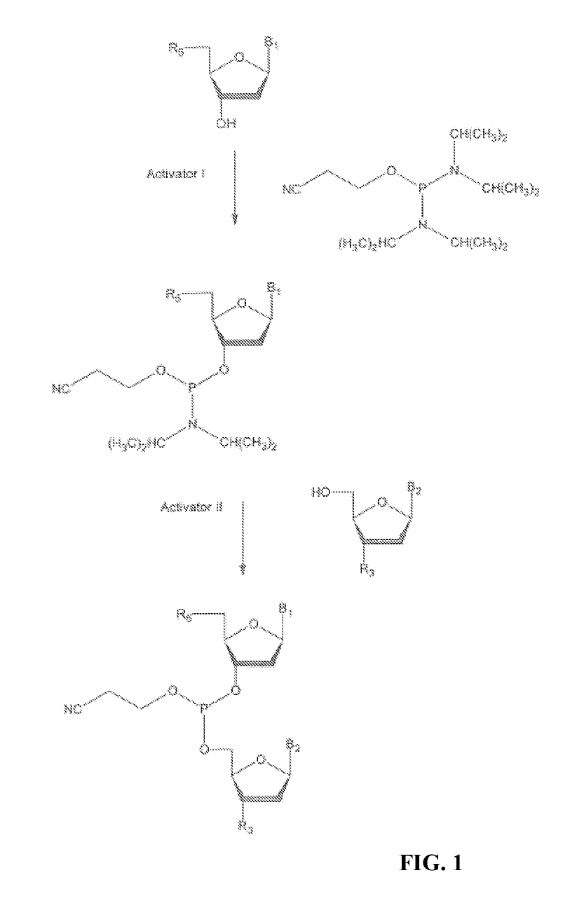

FIG. 1 shows a reaction scheme according to the invention.

Suitable examples of depronated acids B⁻ are trifluoroacetate, triflate, dichloroacetate, mesyl, tosyl, o-chlorophenolate. Acids with a pKa below 4.5 are preferred. Preferably, they have a low nucleophilicity.

In one embodiment, the reaction is conducted in the presence of a molecular sieve to dry the reaction medium. In general, water should be excluded or fixed by drying media during reaction.

It is either possible to combine the activator I of the present invention with the phosphitylating agent and add the hydroxyl component later. It is also possible to combine the activator I with the hydroxyl containing compound and add the phosphitylating agent thereafter.

In the case of using an additive, the activator is mixed with the hydroxyl component before the phosphitylating agent is added.

For the “in situ” generation of the activator the selected acid is preferably added after the addition of the additive under controlled reaction temperature.

The phosphitylating agent can be added before the addition of the selected acid or thereafter.

In relation to the addition of acid and phosphitylating agent the nucleoside component can be added at the end or at the beginning.

In a preferred embodiment, the corresponding base of the activator, the hydroxyl containing compound, and the phosphitylating agent are combined and the acid is added to start the reaction.

The phosphitylated compound (phosphoramidite) is then coupled to a hydroxyl group of a nucleoside, a nucleotide or an oligonucleotide in the presence of activator II.

After reacting a compound as described above, the prepared triesters are oxidized. Oxidation may be used to prepare stable phosphate or thiophosphate bonds, for example.

As used herein oligonucleotides covers also oligonucleosides, oligonucleotide analogs, modified oligonucleotides, nucleotide mimetics and the like in the form of RNA and DNA. In general, these compounds comprise a backbone of linked monomeric subunits where each linked monomeric subunit is directly or indirectly attached to a heterocyclic base moiety. The linkages joining the monomeric subunits, the monomeric subunits and the heterocyclic base moieties can be variable in structure giving rise to a plurality of motives for the resulting compounds.

The invention is especially useful in the synthesis of oligonucleotides having the formula Xₙ, wherein each X is selected from A, dA, C, dC, G, dG, U, dT and n=2 to 30, preferably 2 to 12, more preferably 2 to 8 or 2 to 6 and derivatives thereof comprising protective groups. Modifications known in the art are the modification of the heterocyclic bases, the sugar or the linkages joining the monomeric subunits. Variations of internucleotide linkages are for example described in WO 2004/011474, starting at the bottom of page 11, incorporated by reference.

Typical derivatives are phosphorthioates, phosphorodithioates, methyl and alkyl phosphonates and phosphonoaceto derivatives.

Further typical modifications are at the sugar moiety. Either the ribose is substituted by a different sugar or one or more of the positions are substituted with other groups such as F, O-alkyl, S-alkyl, N-alkyl. Preferred embodiments are 2′-methyl and 2′-methoxyethoxy. All these modifications are known in the art.

Concerning the heterocyclic base moiety, there are a number of other synthetic bases which are used in the art, for example 5-methyl-cytosine, 5-hydroxy-methyl-cytosine, xanthine, hypoxanthine, 2-aminoadenine, 6-alkyl or 2-alkyl derivatives of adenine and guanine, 2-thiouracil. Such modifications are also disclosed in WO 2004/011474 starting from page 21.

When used in synthesis these bases normally have protecting groups, for example N-6-benzyladenine, N-4-benzylcytosine or N-2-isobutyryl guanine.

In general, all reactive groups which are not intended to react in a further reaction have to be protected, especially the hydroxyl groups of the sugar.

In embodiments related to the synthesis of oligonucleotides it is useful to conduct the reaction in the presence of aldehydes or ketones that can be either used as a reaction media or as a co-solvent for other solvents.

Suitable compounds are those that may form enoles. Typical compounds have the formula R₁R₂C═O, wherein R₁ and R₂ are independently H or consist of 1 to 20 carbon atoms which may form cyclic structures alone or R₁ and R₂ form cyclic systems together wherein not both R₁ and R₂ are H. A very preferred ketone is acetone. The presence of acetone quenches the activity of any amount of amines, like diisopropylamine (DIPA), which is liberated during the phosphitylation process. This can be used for the phosphitylation of shorter and longer oligonucleotides with similar results (no decomposition). Other ketone compounds having the formula Rₓ—C(═O)—R_{y} wherein Rₓ and R_{y} are independently C₁-C₆ alkyl or form an cycloalkyl together can also be used as long as they are able to form enolates in the presence of, e.g. amines has a CH₂-group in the α-position.

The invention is further explained by the following non-limiting examples.

### Example 1

5′-O-(4,4′-Dimethoxytriphenylmethyl)-N-isobutyryl-2′-desoxyguanosine (dG-OH) and N-methylimidazolium trifluoroacetate (MIT) were dissolved in acetone and dichloromethane (1:1) and molecular sieve was added. This suspension was added at room temperature to a solution of 2-cyanoethyl N,N,N′,N′-tetraisopropylphosphordiamidite (BisPhos) in dichloromethane with vigorous stirring. A solution of 3′-O-levulinyl-N-isobutyryl-2′-desoxyguanosine (HO-G-1), ethylthiotetrazole (ETT) or imidazolium Trifluoroacetate (IT, CHK346/06) and NMI, dissolved in acetone and dichloromethane (1:1) was added. The reaction was followed by RP-HPLC and after complete conversion, CUROX® M-400 was added. The reaction was followed by RP-HPLC and after complete conversion a filtration step was used to remove the molecular sieve followed by a washing step with acetone/dichloromethane (1:1). The solution was transferred into MTBE to precipitate the reaction product. The precipitate was filtered, washed with MTBE and dried at reduced pressure at 40° C.

| | yield | | d-G-OH | BisPhos | MIT | HO-G-I | ETT/IT |
|---|---|---|---|---|---|---|---|
| Charge | [g] | [%] | [mmol] | [mmol] | [mmol] | [mmol] | [mmol] |
| 1 | 17.94 | 125 | 15.63 | 18.73 | 19.88 | 12.06 | 32.78 |
| 2 | 15.46 | 108 | 15.63 | 17.19 | 1.68 | 12.06 | 28.89 |
| 3 | n.b. | n.b. | 1.56 | 1.72 | 1.84 | 1.21 | 2.23 |
| 4 | n.b. | n.b. | 1.56 | 1.72 | 1.84 | 1.21 | 2.57 |
| 5 | n.b. | n.b. | 1.56 | 1.72 | 1.68 | 1.21 | 2.89 |
| 6 | n.b. | n.b. | 1.56 | 1.72 | 1.68 | 1.21 | 2.89 |
| 7 | n.b. | n.b. | 1.56 | 1.72 | 1.68 | 1.21 | 2.89 |
| 8 | n.b. | n.b. | 1.56 | 1.56 | 1.68 | 1.41 | 3.21 |
| 9 | n.b. | n.b. | 1.56 | 1.40 | 45.89 | 1.57 | 3.21 |
| 10 | n.b. | n.b. | 1.56 | 1.48 | 45.89 | 1.49 | 3.21 |
| 11 | n.b. | n.b. | 1.56 | 1.56 | 40.79 | 1.57 | 3.21 |
| 12 | 57.79 | 124 | 39.08 | 42.98 | 19.88 | 39.16 | 80.28 |
| 13 | 45.65 | 98 | 39.08 | 42.98 | 19.88 | 39.16 | 80.28 |

### Example 2

5′-O-(4,4′-Dimethoxytriphenylmethyl)-N-isobutyryl-2′-desoxyguanosine (dG-OH) and N-methylimidazolium trifluoroacetate (MIT) were dissolved in acetone and dichloromethane (1:1) and molecular sieve was added. At room temperature BisPhos was added under vigorous stirring and a solution of 3′-O-levulinyl-N-isobutyryl-2′-desoxyguanosine (HO-G-1), imidazole und NMI, dissolved in acetone and dichloromethane (1:1) and TFA, dissolved in dichloromethane were added drop wise. The reaction was followed by RP-HPLC. After a complete conversion, CUROX® M-400 was added. Again the reaction was followed by RP-HPLC. After complete conversion, the solution was filtered to remove the molecular sieve, washed with acetone/dichloromethane (1:1) and transferred to MTBE to precipitate the product. The product was filtered, washed with MTBE and dried at reduced pressure by 40° C.

| | | | d-G-OH | BisPhos | MIT | HO-G-I | Imidazole | TFA |
|---|---|---|---|---|---|---|---|---|
| Charge | yield [g] | [%] | [mmol] | [mmol] | [mmol] | [mmol] | [mmol] | [mmol] |
| 14 | 17.25 | 120 | 15.63 | 18.73 | 19.88 | 12.06 | 32.76 | 35.00 |
| 15 | 14.94 | 104 | 15.63 | 18.73 | 19.88 | 12.06 | 18.65 | 43.48 |
| 16 | 20.36 | 118 | 15.63 | 18.73 | 1.68 | 14.47 | 42.58 | 69.60 |
| 17 | 18.44 | 129 | 15.63 | 18.73 | 1.68 | 12.03 | 43.36 | 66.91 |

### Example 3

5′-O-(4,4′-Dimethoxytriphenylmethyl)-N-isobutyryl-2′-desoxyguanosine (dG-OH) and NMI were dissolved in acetone and dichloromethane (1:1) and molecular sieve was added. At room temperature BisPhos was added drop wise and solution of TFA in dichloromethane was added drop wise, too. The reaction was followed by RP-HPLC and after complete conversion a solution of 3′-O-levulinyltymidine (HO-T-1) and imidazole, dissolved in acetone and dichloromethane (1:1) was added. Furthermore, a solution of TFA in dichloromethane was added drop wise. The reaction was followed via RP-HPLC and after complete conversion, CUROX® M-400 was added. Again the reaction was followed via RP-HPLC. After complete conversion, it was filtered to remove molecular sieve washed with acetone/dichloromethane (1:1) and transferred into MTBE to precipitate the product. The precipitate was filtered, washed with MTBE and dried under reduced pressure at 40° C.

| | | | d-G-OH | BisPhos | NMI/TFA | HO-T-I | Imidazole/TFA |
|---|---|---|---|---|---|---|---|
| Charge | yield [g] | [%] | [mmol] | [mmol] | [mmol] | [mmol] | [mmol] |
| 18 | 18.30 | 116 | 15.63 | 17.13 | 31.23/ | 14.39 | 39.22/ |
| | | | | | 20.19 | | 46.45 |
| 19 | 19.46 | 124 | 15.63 | 17.13 | 31.23/ | 14.37 | 39.22/ |
| | | | | | 20.19 | | 46.45 |

1. A method for preparing an oligonucleotide comprising the steps of
a) providing a hydroxyl containing compound having the formula: 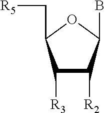
wherein
B is a heterocyclic base; and
wherein
i) R₂ is H, a protected 2′-hydroxyl group, F, a protected amino group, an O-alkyl group, an O-substituted alkyl, a substituted alkylamino, or a C4′-O2′ methylene linkage,
R₃ is OR′₃, NHR″₃, NR″₃R′″₃, wherein R′₃ is a hydroxyl protecting group, a protected nucleotide or a protected oligonucleotide, and wherein R″₃ and R′″3 are independently amine protecting groups,
and R₅ is OH;
or
ii) R₂ is H, a protected 2′-hydroxyl group, F, a protected amino group, an O-alkyl group, an O-substituted alkyl, a substituted alkylamino, or a C4′-O2′ methylene linkage,
R₃ is OH, and
R₅ is OR′₅ and R′₅ is a hydroxyl protecting group, a protected nucleotide, or a protected oligonucleotide;
or
iii) R₂ is OH,
R₃ is OR′₃, NHR″₃, NR″₃R′″₃, wherein R′₃ is a hydroxyl protecting group, a protected nucleotide or a protected oligonucleotide, and wherein R″₃ and R′″₃ are independently amine protecting groups, and
R₅ is OR′₅ and R′₅ is a hydroxyl protecting group, a protected nucleotide or a protected oligonucleotide;
b) reacting said compound with a phosphitylating agent in the presence of an activator having the formula (I) 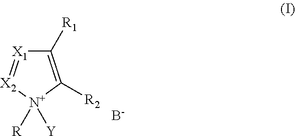
wherein
R=alkyl, cycloalkyl, aryl, aralkyl, heteroalkyl, or heteroaryl;
R₁, R₂=either H or form a 5-membered or 6-membered ring together;
X₁, X₂=independently either N or CH;
Y=H or Si(R₄)₃, wherein R₄=alkyl, cycloalkyl, aryl, aralkyl, heteroalkyl, or heteroaryl; and
B⁻=deprotonated acid;
to prepare a phosphitylated compound;
c) reacting the phosphitylated compound without isolation with a second compound having the formula 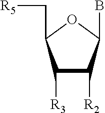
wherein R₅, R₃, R₂, B are independently selected, but have the same definition as above,
in the presence of an activator II selected from the group consisting of imidazole, imidazolium salts, and mixtures thereof.

2. The method of claim 1, wherein the activator (I) has a formula selected from the group consisting of 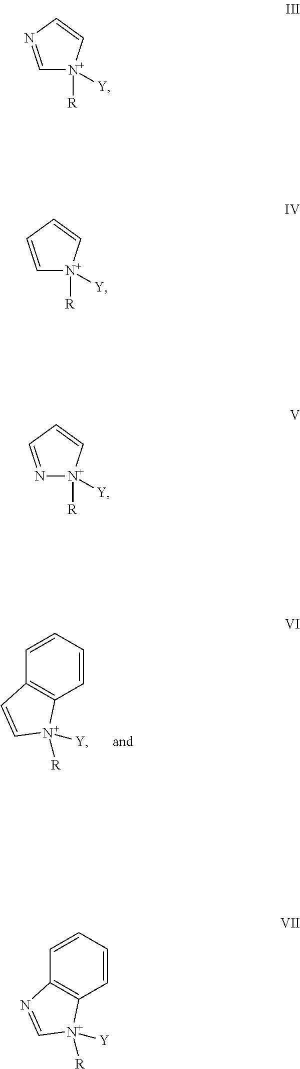
wherein
Y is H or Si(R₄)₃, wherein R₄=alkyl, cycloalkyl, aryl, aralkyl, heteroalkyl, or heteroaryl; and
R is methyl, phenyl, or benzyl.

3. The method of claim 1, wherein the phosphitylating agent has the formula II 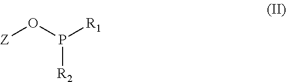
wherein Z represents a leaving group, and wherein R₁ and R₂ are independently secondary amino groups.

4. The method of claim 1, wherein the phosphitylating agent is 2-cyanoethyl-N,N,N′,N′-tetraisopropylphosphorodiamidite.

5. The method of claim 1, wherein the deprotonated acid is selected from the group consisting of trifluoroacetic acid, dichloroacetic acid, methanesulfonic acid, trifluoromethanesulfonic acid, and o-chlorophenolic acid.

6. The method of claim 1, wherein the reacting is in the presence of acetone.

7. The method of claim 1, wherein the concentration of phosphitylating agent in step b) is from 1.0 to 1.2 mol/mol of hydroxyl groups in the hydroxyl containing compound.

8. The method of claim 1, wherein the concentration of phosphitylating agent in step b) is from 3 to 5 mol/mol of hydroxyl groups in the hydroxyl containing compound.

9. The method of claim 1, further comprising adding a polymeric alcohol after step b).

10. The method of claim 9, wherein the polymeric alcohol is polyvinyl alcohol.

11. The method of claim 1, wherein the deprotonated acid is selected from the group consisting of trifluoroacetic acid, dichloroacetic acid, methanesulfonic acid, trifluormethanesulfonic acid (triflate), o-chlorophenolate, and mixtures thereof.

12. The method of claim 9, wherein the reacting is in the presence of acetone.

13. The method of claim 6, wherein the acetone comprises at least 95% (w/w) of the reaction medium.

14. The method of claim 1 wherein the reaction mixture further comprises less then 0.5 mol tetrazole or tetrazole derivatives per mol of the second compound of step c).

15. The method of claim 14, wherein the reaction mixture comprises less than 0.1 mol of tetrazole or tetrazole derivatives per mol of the second compound of step c) or no tetrazole or tetrazole derivatives.
